(19) United States
(12) Patent Application Publication
(10) Pub. No.: US 2025/0380943 A1
(43) Pub. Date: Dec. 18, 2025

(54) Suture Constructs and Methods of Tissue Fixation

(71) Applicant: Arthrex, Inc., Naples, FL (US)

(72) Inventors: Joshua BEST, Naples, FL (US); Sarah A. MUEHLEMAN, Tampa, FL (US); Tara L. SWANLAW, Fort Myers, FL (US); Zachary A. INGWER, Naples, FL (US); Justin M. BOYLE, Naples, FL (US); Eric S. ZAJAC, Naples, FL (US)

(73) Assignee: Arthrex, Inc., Naples, FL (US)

(21) Appl. No.: 18/867,469

(22) PCT Filed: Jun. 14, 2023
(86) PCT No.: PCT/US2023/025242

§ 371 Date: Nov. 20, 2024

Related U.S. Application Data
(60) Provisional application No. 63/352,666, filed on Jun. 16, 2022.

(51) Int. Cl. A61B 17/06

(52) U.S. Cl. CPC A61B 17/06166; A61B 2017/06185

(57) ABSTRACT

Knotless constructs and methods of tissue repairs are disclosed herein. A suture ring can be formed of suture with a hollow core. A suture ring can be folded to a “S”, “L′”, or “W” configuration. A suture ring can be loaded with a tensionable mechanism to assist in knotless, tensionable tissue repairs.

### BACKGROUND

The disclosure herein relates to surgical constructs and, more specifically, to knotless suture constructs and associated methods of tissue repairs.

### SUMMARY

Knotless constructs, surgical systems, assemblies, and methods of tissue repairs are disclosed. A construct can create a knotless repair. In an embodiment, a construct as described herein can be self-locking. A soft tissue repair system includes a tensionable construct with a fixation device in the form of a suture ring pre-loaded with a flexible coupler and a shuttle/pull device attached to the flexible coupler. A construct can include a plurality of interconnected adjustable loops. A flexible coupler can be tape such as suture tape. A suture ring can be flexible. A suture ring can be formed of suture with a hollow core. A suture ring can include braided strands of suture and at least one radiopacifier. A suture ring can include braided suture of ultrahigh molecular weight polyethylene and polyester. A suture ring can consist essentially of ultrahigh molecular weight polyethylene suture and bismuth trioxide.

Methods of tissue repairs are also disclosed. A first tissue is approximated to a second tissue with a knotless surgical construct that includes at least one tensionable construct with a mechanism. One of two free ends of a flexible coupler (suture or tape) is passed at different locations through a body of a fixation device (a folded flexible soft suture ring). The two ends of the flexible coupler form adjustable, closed, tensionable, flexible loops that are spliced and are interconnected. The ends can be pulled to tension and lock the construct.

### BRIEF DESCRIPTION OF THE DRAWINGS

FIGS. 1-6 illustrate various views of a fixation device.

FIG. 7 illustrates a schematic side view of a flexible coupler that forms fixation device of FIG. 1.

FIG. 8 illustrates a side view of the fixation device of FIG. 1.

FIGS. 9-12 illustrate subsequent steps for the formation of a surgical assembly according to an exemplary embodiment.

FIG. 13 illustrates another surgical assembly with the fixation device of FIG. 1.

FIGS. 14 and 15 illustrate another surgical assembly with the fixation device of FIG. 1.

FIGS. 16-19 illustrate additional views of the surgical assembly of FIG. 12.

FIG. 20 illustrates the surgical assembly of FIG. 12 employed in ACL reconstruction.

FIG. 21 illustrates another fixation device.

### DETAILED DESCRIPTION

The disclosure provides surgical adjustable loop constructs, suture loop mechanisms, and methods for securing a first tissue to a second tissue (for example, soft tissue to bone, or bone to bone) with a tensionable construct including adjustable, knotless, flexible, closed loops.

The surgical construct can be a suture with a hollow core and picks per inch (ppi) of 52 or less. The suture can have 40 ppi or less. The suture can have 25 ppi or less. The suture can have 15 to 20 ppi. The suture can have 18 ppi. The suture can have 32 yarns or less.

In an embodiment, the suture construct has a ppi of 25, 24, 23, 22, 21, 20, 19, 18, 17, 16, or 15. In an embodiment, the suture construct is an enclosed ring that is configured to fold into a “S”, “U”, or “W” configuration. Embodiments of the suture construct can be attached to another suture construct (e.g., TightRope® construct) with a tissue graft. The suture construct as described herein can fix the construct on a bone, wherein the “S”, “U”, or “W” configuration is flush on the surface of the bone with a relatively low profile. Such a suture construct seated on a bone will fully seal off a bone tunnel and lock in biologics. Thus, the suture construct as described herein used as a fixation device on the surface of the bone can lead to faster healing. Such a suture construct can be used instead of a metal button, thereby, being safer for use with MRIs and being more biomimetic.

In one embodiment, the disclosure provides a suture construct. An orthopedic implant construct is utilized to attach or re-attach normal anatomical structures, tissue to tissue, bone to bone, and/or bone to soft tissue. The construct can be composed of one single continuous flexible coupler in the form of suture, round, and/or flat suture attached to a suture ring. The flexible coupler forms a plurality of knotless, continuous, flexible, closed adjustable loops having an adjustable perimeter. Terminal ends of the flexible coupler exit the suture ring. The construct can be shrunk when both terminal ends are pulled. When the terminal ends are pulled, the construct shrinks, i.e., the perimeters of the knotless, continuous, flexible, closed adjustable loops decrease. The tensionable construct allows the user (for example, surgeon) to control the tension of the flexible coupler on first tissue (for example, soft tissue) to be attached to a second tissue (for example, bone).

A soft tissue repair system includes a fixation device in the form of a soft, flexible suture button; a flexible coupler with two flexible ends (a first end and a second end) wherein the first end forms a first splice and a loop; a suture passer attached to the loop; and a shuttle/pull device attached to the second end of the flexible coupler. A plurality of interconnected, adjustable, continuous, flexible loops is formed by passing the suture passer through the fixation device so that the first end of the flexible coupler exits the fixation device; passing the second end of the flexible coupler through the loop at the first end and then through an eyelet of the shuttle/pull device; pulling on the shuttle/pull device to pass the second end through the flexible coupler and form a second splice; centering the fixation device relative to the first and second splices; and pulling on the first end and the second end to tension and lock the construct. A flexible coupler can be suture or suture tape. A suture button can be an implantable suture button or a cortical suture button. A suture button can be formed of a suture with a hollow core having a ppi of 52 or less, and a yarn count of 32 or less. A suture button can include braided strands of suture with one or more radiopacifiers. A suture button can include braided suture of ultrahigh molecular weight polyethylene and polyester. A suture button can consist essentially of ultrahigh molecular weight polyethylene suture and radiopaque material, such as bismuth trioxide and barium sulfate.

A flexible coupler forms a plurality of closed, knotless, continuous, adjustable, flexible loops having adjustable perimeters, the loops being located between a loop interconnection and two terminal ends. A suture ring is provided at the terminal ends and adjacent each closed, knotless, continuous, adjustable, flexible loop. A construct can be knotless. A construct can be employed to re-attach normal anatomical structures, i.e., a first tissue to a second tissue, such as soft tissue, tendon, ligament, and/or bone, to each other and/or any combination of one another, by employing a mechanism. A flexible coupler can be suture and/or tape. A construct can be employed as a stand-alone construct or with additional fixation devices, for example, attached to an additional implant, anchor, screw, plate, button (such as a metal button or another suture ring), etc.

A suture button construct for knotless repairs is disclosed. The suture construct includes a flexible coupler of either round or flat design that is run through a folded suture ring. The flexible coupler is passed through the folded suture ring and then through itself creating two interconnected, flexible, continuous, adjustable, knotless, closed suture loops with adjustable perimeters. The suture ring can be secured to tissue. Tension can be applied to ends of the flexible coupler to shrink/close the loops bringing the suture ring and tendon/ligament/and/or soft tissues together while locking the construct in place. The suture ring can include braided strands of suture material with one or more radiopacifiers. The suture ring can include braided suture of ultrahigh molecular weight polyethylene and polyester. The suture ring can consist essentially of ultrahigh molecular weight polyethylene suture and Bismuth Trioxide. The suture ring can be formed of a suture with a hollow core and having a ppi of 52 or less. The suture ring can be formed of a suture with a hollow core and 32 yarns or less.

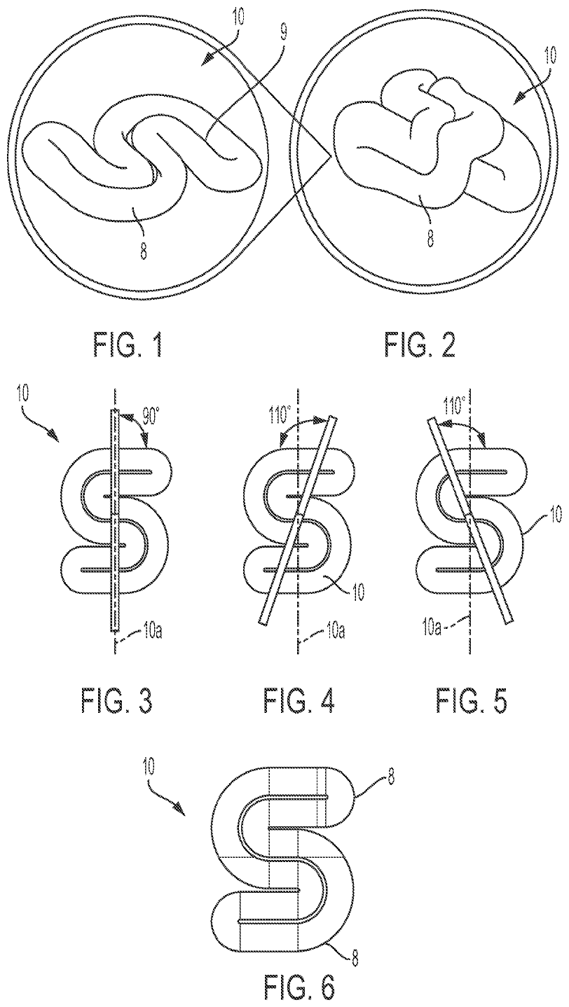

Referring now to the drawings, where like elements are designated by like reference numerals, FIGS. 1-21 illustrate structural elements of surgical assembly 100, 200, 300 (surgical construct 100, 200, 300; construct 100, 200, 300; knotless construct 100, 200, 300; knotless flexible button construct 100, 200, 300) formed of tensionable construct 20, 30, 40 attached to fixation device 10.

Fixation device 10 (suture ring 10; ring of suture 10; FiberRing™ suture 10; FiberRing™ 10; ring 10; suture button 10; folded suture ring 10; anchor 10; soft anchor 10; implant 10; flexible button 10) is illustrated in FIGS. 1-9.

Fixation device 10 is a soft flexible suture button in the form of a ring. In an embodiment, fixation device 10 is a ring formed essentially of suture. In an embodiment, fixation device 10 is formed of suture comprising a hollow core and 52 picks per inch or less in an enclosed ring. The suture can have 40 ppi or less. The suture can have 25 ppi or less. The suture can have 15 to 20 ppi. The suture can have 18 ppi. The suture can have 32 yarns or less. In an embodiment, the suture construct has a ppi of 25, 24, 23, 22, 21, 20, 19, 18, 17, 16, or 15. In an embodiment, the suture construct is an enclosed ring that is configured to fold into a “S”, “U”, or “W” configuration.

In an embodiment, fixation device 10 is a ring formed of braided suture. In an embodiment, fixation device 10 is a ring formed of braided suture strands and at least one radiopacifier. In an embodiment, braided suture can include fibers of ultrahigh molecular weight polyethylene (UHMWPE) braided with polyester. UHMWPE is easy to splice and pierce; allows the construct to deform and remain in deformed shape; even at high PPI, it has minimal fraying and snagging; and has a low coefficient of friction. Polyester is difficult to splice without fraying suture; allows the construct to retain initial form; difficult to pierce and pass TightRope® suture without fraying and snagging; and has a high coefficient of friction.

In an embodiment, fixation device 10 is an all-suture ring braided from fibers of UHMWPE with a radiopaque material. In an embodiment, the radiopaque material can be bismuth trioxide, barium sulfate, iodine, nickel, nitinol, and combinations thereof. The size of the fibers, braiding density and loop length can be optimized to create a low-profile button with equivalent performance to metal counterparts. In an embodiment, the concentration of the sample is of about 50% mixture; however the optimal amount of UHMWPE with bismuth trioxide depends upon the indication and placement of the anchor in the body. In an embodiment, fixation device 10 can include braided suture of ultrahigh molecular weight polyethylene and polyester. In an embodiment, fixation device 10 can consist essentially of ultrahigh molecular weight polyethylene suture and bismuth trioxide. In an embodiment, fixation device 10 can consist of ultrahigh molecular weight polyethylene suture and bismuth trioxide.

Referring now to FIGS. 3-5, fixation device 10 can be any device that allows passing of flexible couplers therethrough (for example, through a plurality of locations formed within and/or along a body of the fixation device) to form at least one flexible, adjustable loop. In an exemplary embodiment, the fixation device 10 is a suture ring 10 having an S-shaped configuration that allows passage of a flexible coupler 11 (and optional formation of loops and interconnection). Fixation device 10 (suture ring 10) can be folded into three sections to achieve an exemplary “S” configuration and secure for suture passage. Fixation device 10 can be pierced by flexible coupler 11 at various angles and/or positions with respect to longitudinal axis 10a of the ring, to achieve the exemplary configurations of FIGS. 3-5.

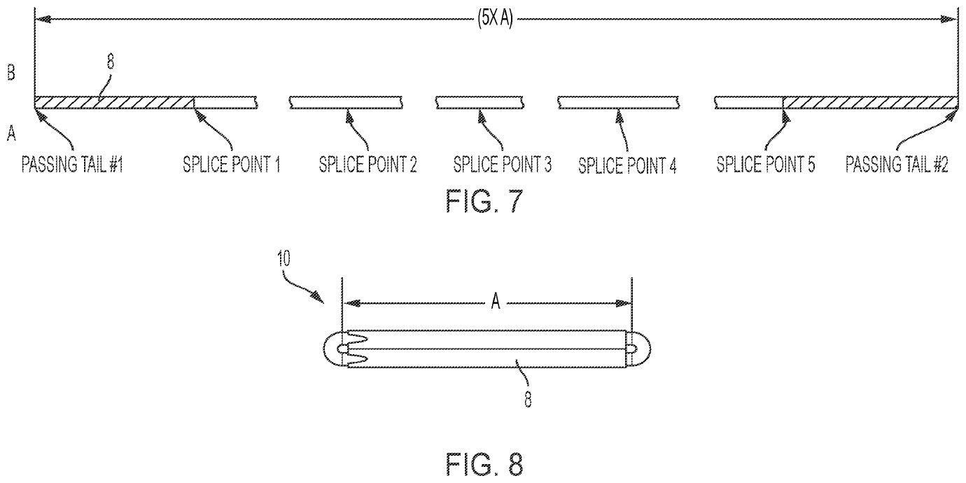

FIGS. 7 and 8 schematically illustrate a method of forming a ring 10 of suture 8. Ring 10 can be created from a loop 9 of suture 8 that is folded and then pierced to create a complex structure with a configurable packing factor. Suture 8 can be marked at 5 positions: splice point 1; splice point 2; splice point 3; splice point 4; and splice point 5. Passing tails 1 and 2 are provided on each side of suture 8 to aid in the formation of ring 10. A method of forming ring 10 can include the following steps: inserting passing tail 1 into splice point 4 on side A; direct passing tail 1 to splice point 3; exiting passing tail 1 at splice point 3 on side B; inserting passing tail 2 to splice point 3 on side B; direct passing tail 2 to splice point 3; and exiting passing tail 2 at splice point 3 on side B. The method can further include folding the ring 10 into three sections to form a “S”. “U”, or “W” configuration. Ring 10 has opposing splices that allow tightening and locking up as passing tails 1, 2 are pulled.

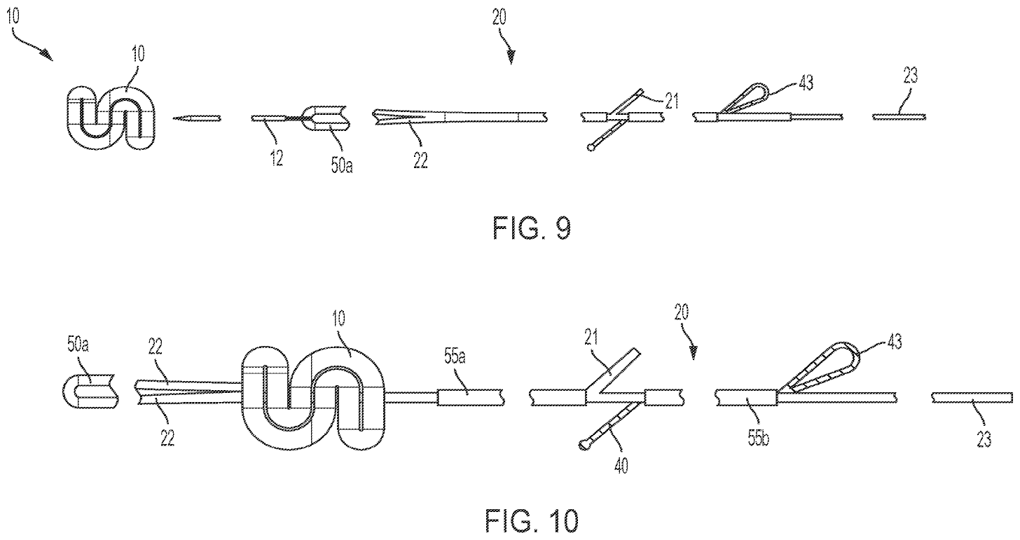

Reference is now made to FIGS. 9-19 which illustrate suture ring 10 employed with additional elements to form assemblies 100, 200, 300. The additional, adjoining elements can be in the form of tensionable constructs. The additional elements can include a TightRope® device, FiberWire® and TigerWire® sutures for passing, and FiberTape® suture for InternalBrace® fixation of bone to bone or soft tissue to bone, and are intended as fixation posts, a distribution bridge, or for distributing suture tension over areas of ligament or tendon repair.

FIGS. 9-12 illustrate exemplary steps of assembling fixation device 10 (suture ring 10) with tensionable construct 20 to form assembly 100 (FIG. 12). Tensionable construct 20 can be an exemplary TightRope® construct. The assembly process can begin by passing tensionable construct 20 attached to suture passer 12 through suture ring 10. Suture passer 12 can be a needle with a passing loop such as a nitinol loop, for example, or any similar suturc passing device. FIG. 9 illustrates fixation device 10 and tensionable construct 20 in unassembled state. FIGS. 10-12 illustrate fixation device 10 and tensionable construct 20 in assembled state.

Tensionable construct 20 can be formed of one single continuous flexible coupler 22 (coupler 22; flexible material 22; flexible strand 22; flexible tape 22) in form of suture, either round and/or flat suture, for example, suture tape. The flexible coupler 22 is provided with two terminal ends, a first end 21 and a second end 23. One of the two terminal ends 21, 23 (for example, first end 21) is spliced at first splice 55a to form a first suture loop 50a. The other of the two terminal ends 21, 23 (for example, second end 23) has a shuttle/pull device 40 spliced thereon at a second splice 55b. Exemplary shuttle/pull device 40 can be in the form of a suture passing instrument or a suture passer such as FiberLink™ 40 or nitinol loop 40. Suture passing device 40 includes an eyelet/loop 43 for passing the flexible coupler 22.

FIG. 10 illustrates tensionable construct 20 passed through body 11 of fixation device 10 and with suture passer 12 removed from first suture loop 50a. Suture passer 12 can be removed once loop 50a is passed through the far side (distal side) of fixation device 10.

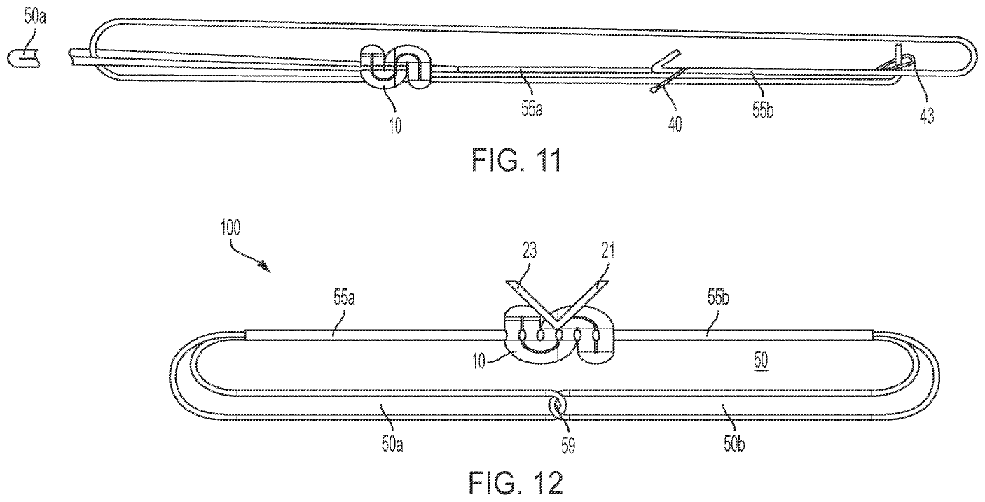

FIG. 11 illustrates loading of the long free tail 23 (second end 23) of flexible coupler 22 through the open loop 50a. The long free end 23 is then passed through the eyelet 43 of the shuttle/pull device 40. The shuttle/pull device 40 is pulled out of the flexible coupler 22 pulling the long end 23 through the second splice region 55b and forming a second loop 50b. The fixation device 10 (FiberRing™ 10) can then be open by pulling the center apart and the splices 55a, 55b and tails 21, 23 are centralized (FIG. 12). Ring 10 is closed by pushing the ends of the ring together. Splices 55a, 55b are locking splices. The length of each locking splice 55a, 55b can be variable (at least about 3 mm up to the length of ring 10). The distance between the splices 55a, 55b can be variable, for example, between about 0 mm to about 3 mm. The termination of splices 55a. 55b can either reside within the inner lumen or exit the lumen (hollow core). Further, in lieu of a double locking splice, a static knot can be formed, and the tails can be tucked within the corresponding lumens.

As shown in FIG. 12, the loops 50a, 50b are connected by interconnection 59 (loop interconnection 59 or intertwined region 59) formed by the flexible coupler 22 and forming, therefore, another flexible loop 50. The at least two loops 50, 50a, 50b are flexible, closed, knotless, continuous, adjustable loops each having an adjustable perimeter. Interconnection 59 is located at one end of the assembly 100 while the fixation device 10 (FiberRing™ 10) is located at another end of the assembly 100 (for example, an end opposite the one end). Surgical assembly 100 is a tensionable construct that can be shrunk when both terminal ends 21, 23 are pulled to decrease the perimeter of at least one of the flexible, closed, knotless, adjustable loops 50, 50a, 50b. Additional flexible strands can be attached to assembly 100 by passing the flexible strands through any of the loops 50, 50a, 50b, 9. Additional strands can be FiberWire® suture, TigerWire® suture, FiberTape® suture tape, among many others.

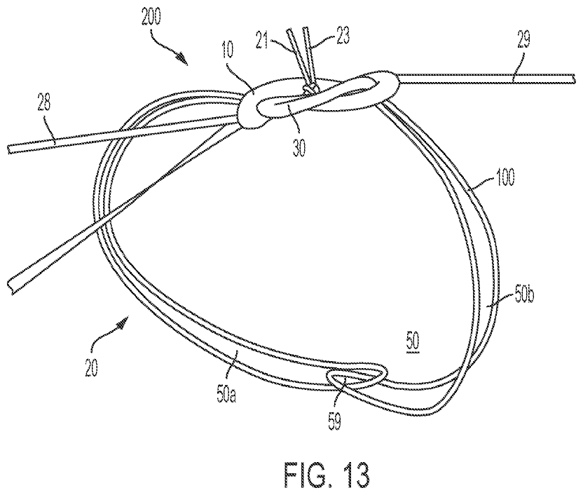

Assembly 100 can be employed by itself or in conjunction with additional structures and/or fixation devices, for example, additional flexible couplers, anchors, implants, and/or buttons, to form additional surgical constructs. For example, FIG. 13 illustrates assembly 200 which includes assembly 100 with an additional flexible coupler 30 in the form of FiberTape® suture tape for an InternalBrace™ procedure. Assembly 200 also includes two passing sutures 28, 29 (passing tail 28 and passing tail 29).

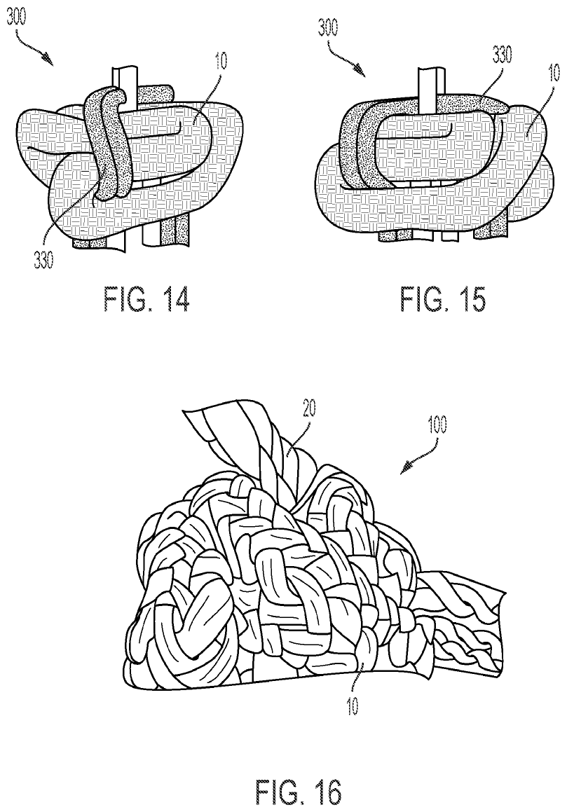

FIGS. 14 and 15 illustrate assembly 300 with fixation device 10 (folded anchor construct 10) with sutures 330 in the form of FiberTape® suture tapes 330 of an InternalBrace™ procedure.

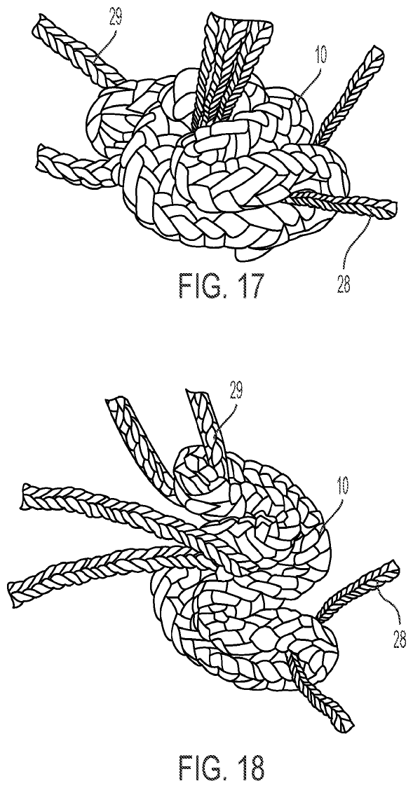

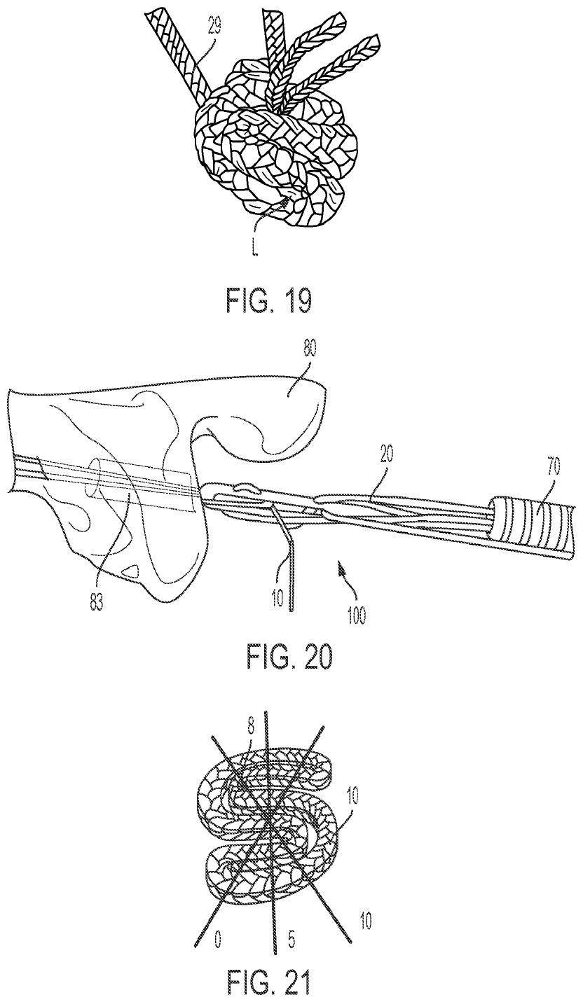

FIGS. 16-19 illustrate additional views of assembly 100 of FIG. 12. FIG. 16 shows a direct side view of implant 100 tensioned over a bone hole (for example, a 3.5 mm hole), demonstrating the legs of the button construct moving over top of the tensioning hole. FIG. 17 is an angled isometric view detailing the control of deformation after releasing tension from the blue and white passing sutures. FIG. 18 is a top down view detailing the “S” configuration prior to loading of the TightRope® construct to pull the legs underneath the top strands of the button. FIG. 19 is an angled top down view detailing the removal of one of the passing sutures (passing suture 28) to show location L where the InternalBrace 30 (flexible coupler 30) can be routed.

Assemblies 100, 200, 300 of FIGS. 1-19 are adjustable loop constructs. Fixation device 10 of assemblies 100, 200, 300 can be a suture button/ring construct in the form of an orthopedic implant construct which can be utilized to attach or re-attach a first tissue to a second tissue, for example, normal anatomical structures, bone to bone, tissue to tissue, and/or bone to tissue, among others.

FIG. 20 illustrates an exemplary method of tissue repair with surgical constructs of the disclosure. Exemplary assembly 100 (surgical construct 100) with fixation device 10 (FiberRing™ 10) and tensionable construct 20 is employed in an exemplary ACL reconstruction. Exemplary graft 70 is looped over interconnection 59. Graft 70 can be an ACL or PCL graft, for example. Surgical construct 100 attaches first tissue (for example, graft 70) to a second tissue (for example, femur 80). Surgical construct 100 extends within a femoral tunnel 83 formed within femur 80 and allows graft 70 to be passed through loop 50 and over loop interconnection 59 of the construct and be further secured within a tibial tunnel or socket formed within tibia. Fixation device 10 (FiberRing™ 10) can be passed through femoral tunnel 83 and exits the femoral cortex to rest upon it.

Femoral socket 83 of femur 80 can be drilled transtibially, or through the medial portal, or by a retrograde technique. The femoral socket 83 is drilled in femur 80 to a depth about equal to the amount of graft desired in the femoral socket. After creating tibial tunnel in tibia (not shown), fixation device 10 (FiberRing™ 10) of assembly 100 is pulled through the femur 80 until it exits the lateral cortex to achieve fixation. The graft is advanced and tension is pulled on the terminal ends 21, 23. The terminal ends can form a knot and/or can be cut with a cutting instrument such as an arthroscopic cutter. The technique proceeds with tibial fixation with another fixation device, for example, an interference screw or another fixation device 10 (second FiberRing™ 10) of another assembly 100. Assembly 100 can be also employed for all-inside ACL reconstruction. The adjustability of the implant simplifies graft length determination and allows graft tensioning from the femoral side.

The flexible loops of the assembly 100 (tensionable construct 100) are adjustable under tension when the surgeon simply pulls on both terminal ends of the final construct 100 to adjust the length of the flexible loops and to tighten, therefore, the construct. The fixation device 10 (FiberRing™ 10) is pulled out of the bone cortex with the passing sutures (which are later discarded) and can flip onto the cortex immediately upon exiting.

The ACL reconstruction detailed above offers adjustable cortical fixation for cruciate ligament reconstruction in a self-locking manner. The self-locking mechanism of the knotless construct 100 resists cyclic displacement and offers maximum loads equal to closed loop devices. The present disclosure eliminates the need for metal fixation devices and facilitates complete graft fill of bone tunnel aperture closures that are common with anatomic ACL drilling.

The ACL reconstruction construct above preferably includes a suture tape such as an ACL TapeRope. The device is used to reattach soft tissue to bone, bone to bone, ligament and/or tendon to bone. As detailed above, the device includes a flat tape (suture tape) run through a flexible suture button. One continuous loop is threaded through the suture button and interwoven back through itself to create a self-locking adjustable loop construct. The device may be provided assembled, disassembled, with or without additional fixation devices and flexible couplers, to facilitate passing through a bone plug, and/or being passed prior to attachment of button(s).

Fixation device 10 in the form of a flexible ring can be folded and compressed from a first configuration (a round or non-folded configuration) to a second configuration (an S-shaped or folded configuration); however, it must be understood that this exemplary-only “S” shape is a non-limiting example, and fixation device 10 can be folded/changed to structures having other shapes and configurations. To maximize the amount of suture material fitting into a small space on top of a bone opening, folding of the hollow suture ring can be conducted to control the dimensions of the folded ring (the folded ring must be small enough to reside on top of the bone yet large enough to avoid passing through the bone opening (hole or tunnel)). Thus, the ring can be also folded into a “U” or “W” configuration, in addition to the “S” configuration, for considerable packing factor.

The use of a loop in assemblies 100, 200, 300 maintains the passing and shuttling sutures typically used in rigid anchor implantation allowing for precise positioning and anchor placement control. The novel suture button 10 can be employed for suspensory fixation as prior art demonstrates use of suture anchor in a bone tunnel or socket and in soft tissue. Anchoring on top of bone can be achieved with the flexible button configuration resisting pull through on top of a rigid hole. The flexible button 10 expands horizontally versus vertically compared to other button designs. Prior art button designs formed large “knots” palpable under the skin. In contrast, the flexible button of the present disclosure allows for specific tailoring of height relative to the bone surface.

Traditional metal, rigid cortical suspensory fixation devices are constructed of titanium. The present disclosure utilizes flexible braided strands of polymer to create a soft, flexible button. The utilization of a suture material allows for designed button deformation resulting in a high degree of control relative to construct strength and clinical performance. Specifically, the design element allowing such increased anchor placement control is the continuous loop of suture which is then folded and secured to the adjoining/additional elements. Further, the material selection of the button 10 allows for the use of radiopacifiers (i.e., materials with a higher electron density contrast compared to the surrounding material so that it absorbs X-ray energy) to be used as fillers within the polymer construction, allowing for custom tailoring of the contrast and sharpness of the construct under fluoroscope or x-ray imaging. The benefits of this construct to the patient include no prominent metal hardware left in the body that can cause soft tissue irritation; less scar tissue formation; less risk of IT band entrapment; and immediate enclosure of the bone tunnel allowing faster healing.

In an embodiment, suture button 10 is a soft, flexible, all-suture button which is braided from fibers of UHMWPE with bismuth trioxide. The size of the fibers, braiding density and loop length are optimized to create a low-profile button with equivalent performance to metal counterparts. In an embodiment, the concentration of the sample is of about 50% mixture; however the optimal amount of UHMWPE with bismuth trioxide depends upon the indication and placement of the anchor in the body. In an embodiment, suture button 10 is formed of suture with a hollow core, 52 picks per inch or less, and 32 yarns or less.

Fixation device 10 can be any device that allows passing of flexible couplers therethrough (for example, through a plurality of locations formed within and/or along a body of the fixation device) to form a plurality of continuous, knotless, flexible, adjustable loops. In an exemplary embodiment, the fixation device is a suture ring having an S-shaped configuration that allows passage of a flexible coupler (and optional formation of loops and interconnection).

Although the embodiments above have been described with reference to a particular ACL reconstruction technique, the disclosure is not limited to this exemplary embodiment. Accordingly, the present disclosure also contemplates embodiments wherein a suture button implant and novel mechanism of the present disclosure is employed for additional tissue positioning and/or tissue adjustment applications, for example, in fixation of bone to bone (such as small joint applications, or acromioclavicular joint fixation techniques) which employ two fixation devices (for example, two flexible suture buttons) joined by a continuous suture loop formed by a continuous flexible coupler. In these applications, a second fixation device (for example, a second suture button) can be used in conjunction with the first suture button and with the flexible coupler, and with additional flexible, tensionable loops between the two suture buttons, to complete the self-locking repair.

In exemplary embodiments only. fixation device 10 of assembly 100, 200, 300 of the present disclosure can be employed in a method of bunion repair and/or in a method of Lisfranc repair. Similarly, fixation device 10 of assembly 100, 200, 300 can be employed in a method of fixation of bone.

Fixation device 10 of assembly 100, 200, 300 can create knotless self-locking repairs. A self-locking suture ring implant is provided for self-locking soft tissue repairs, for example, for self-locking tendon reattachment. A self-locking device incorporating a locking mechanism (knotless flexible, adjustable, continuous, uninterrupted tape suture loop locking mechanism) to lock flexible strands, particularly suture tapes, is also disclosed. The locking mechanism can be employed with any suture tape, i.e. SutureTape, LabralTape, FiberTape®, etc.

The self-locking suture ring implant and the self-locking mechanism can be utilized to attach soft tissue (tendon) to bone such as in shoulder repair or fixate a graft in ACL or PCL reconstruction, for example, in a self-locking manner. Thus, the self-locking suture ring implant and the self-locking mechanism can be utilized in surgical procedures such as rotator cuff repair, Achilles tendon repair, patellar tendon repair, ACL/PCL reconstruction, hip and shoulder reconstruction procedures, AC joint reconstruction, syndesmosis reconstruction, quad/patellar tendon rupture repair, hallux-valgus repair, proximal and/or distal biceps tendon repair, humerus and radius repair, and any other tendon repair to bone, among many others, all conducted in a self-locking manner. Any of tensionable construct 20, 30, 330; fixation device 10 (FiberRing™ 10); and surgical assembly 100, 200, 300 can be employed in the methods of self-locking repairs.

A surgical assembly 100, 200, 300 includes a fixation device 10 and a knotless, adjustable, self-locking tensionable construct 20, 30, 330 pre-loaded on the fixation device 10. Tensionable construct 20 includes at least one flexible coupler 22 having a first end 21 and a second end 23; a loop interconnection 59 between the first end 21 and the second end 23; first and second closed, adjustable, continuous, flexible loops 50a, 50b. Fixation device 10 is a flexible suture ring having an S configuration adjacent the first and second closed, adjustable, continuous, flexible loops 50 and adjacent the first and second ends 21, 23.

A surgical assembly 100 comprises: a flexible coupler 22 passed through a body of a fixation device 10 in the form of a suture ring 10 to form at least two flexible, adjustable, closed, knotless loops 50a, 50h with adjustable perimeters; a loop interconnection 59; and two terminal ends 21, 23. The flexible coupler 22 can be a suture tape and the fixation device 10 can be an implantable ring formed of UHMWPE suture braided with polyester, for example, bismuth trioxide. The flexible coupler 22 can connect a first tissue to a second tissue. The first tissue can be bone 80 and the second tissue can be soft tissue 70. Soft tissue 70 can be attached to the loop interconnection 59, and bone 80 can be attached to the fixation device 10. The terminal ends 21, 23 are pulled to decrease the distance between the fixation device 10 and the soft tissue 70 and decrease the length and perimeter of the flexible, adjustable, closed, knotless loops 50, 50a, 50b.

A self-locking tensionable assembly 100 comprises: a flexible coupler 22 having a first end 21 and a second end 23; a loop interconnection 59 between the first end and the second end; first and second closed, adjustable, continuous, flexible loops 50a, 50b; first and second splice regions 55a, 55b; and a flexible suture ring 10 adjacent the adjustable, continuous, flexible loops 50a, 50b, the first and second splice regions 55a, 55b, and the first and second ends 21, 23. The flexible suture ring, the first and second splice regions, and the first and second ends are located on an end of the construct opposite to the end with the loop interconnection. The flexible suture ring 10 is located between the first and second splice regions 55a, 55b. The flexible coupler 22 is suture, suture tape or ribbon. The flexible coupler 22 is a suture tape with a plurality of sections of cross-sections and/or different tapers. The flexible coupler 22 is a suture tape with round suture and flat suture tape. The first and second ends 21, 23 are pulled to lock the tensionable construct. A tensionable construct 20 can consist essentially of the flexible coupler 22. The flexible suture ring 10 can consist essentially of a braided suture ring. The flexible suture ring 10 can consist essentially of a suture 8 with a hollow core and 52 picks per inch or less in an enclosed ring, wherein the flexible ring 10 is configured to fold into a “S”, “U”, or “W” configuration. The suture can include 32 yarns or less. The flexible suture ring 10 can include suture strands braided with polyester. The flexible suture ring 10 can consist essentially of UHMWPE suture and bismuth trioxide. The fixation device 10 is an implantable button and the flexible coupler 22 is a suture tape. Each terminal end is configured to be pulled to lock the surgical construct. Each terminal end is configured to be pulled to decrease a length and perimeter of at least two flexible, continuous, closed, adjustable, knotless loops 50a, 50b. The tissue repair is rotator cuff repair. AC joint repair, syndesmosis repair, Achilles tendon repair, patellar tendon repair. ACL/PCL reconstruction, hip and shoulder reconstruction. AC joint reconstruction, syndesmosis reconstruction, quad/patellar tendon rupture repair, or hallux-valgus repair.

A method of tissue repair comprises: passing a flexible coupler 22 through different regions of a flexible suturc ring 10 having an S-shaped configuration; passing one of the terminal ends 21, 23 of the flexible coupler 22 through a loop 50a of the flexible coupler 22 and then through an eyelet 43 of a shuttle/pull device 40 attached to the flexible coupler 22 to form at least two flexible, knotless, continuous, closed adjustable loops 50, 50a, 50b with an adjustable perimeter and a loop interconnection 59; attaching a first tissue 70 to the loop interconnection and/or to the flexible coupler; securing the fixation device 10 to a second tissue 80 (for example, bone 80); and pulling on the terminal ends 21, 23 to lock the flexible coupler 22.

A method of forming a knotless self-locking repair comprises: attaching a flexible coupler 22 with a first end 21 and a second end 23 to a fixation device 10 by passing one end 21 of the first and second ends 21, 23 through the fixation device 10 with a first passing device 12, wherein the other end 23 of the first and second ends 21, 23 is attached to a second passing device 40; passing the other end 23 of the first and second ends 21, 23 through first loop 50a of the first end 21 and through eyelet 43 of second passing device 40; pulling second passing device 40 out of the second end 23 to pull the second end 23 through the flexible coupler 22 and form a plurality of flexible, continuous, closed, adjustable, knotless loops 50, 50a, 50b; and centering the fixation device 10 relative to the first and second ends 21, 23, and relative to first and second splice regions 55a, 55b. The flexible, continuous, closed, adjustable, knotless loops 50a, 50b are separated by a loop interconnection 59. At least one of the first and second passing devices 12, 40 is a suturing device. The suturing device can be a needle. The method can further comprise: attaching a first tissue 70 to the flexible coupler 22; attaching the fixation device 10 to a second tissue 80; pulling on the first and second ends 21, 23 of the flexible coupler 22 to adjust tension of the first and second flexible, continuous, closed, adjustable, knotless loops 50a, 50b, to approximate the first tissue 70 to the second tissue 80. The first tissue can be soft tissue and the second tissue can be bone.

A method of forming a ring 10 of suture 8 comprises: inserting passing tail 1 into splice point 4 on side A; direct passing tail 1 to splice point 3; exiting passing tail 1 at splice point 3 on side B; inserting passing tail 2 to splice point 3 on side B; direct passing tail 2 to splice point 3; and exiting passing tail 2 at splice point 3 on side B. The method can further include folding the ring 10 into a plurality of sections to form a construct with a “S”, “U”, or “W” configuration.

The disclosed constructs and surgical assemblies utilize flexible braided strands of polymer to create a soft, flexible suture button 10. The utilization of a suture material allows for designed button deformation resulting in a high degree of control relative to construct strength and clinical performance. Specifically, the design element allowing such control is the continuous loop of suture which is then folded and secured to the adjoining elements.

Further, the material selection of the button allows for the use of radiopacifiers to be used as fillers within the polymer construction allowing for custom tailoring of the contrast and sharpness of the construct under fluoroscope or x-ray imaging. The benefits of this construct to the patient include no prominent metal hardware left in the body that can cause soft tissue irritation, less scar tissue formation, less risk of IT band entrapment, and immediate enclosure of the bone tunnel allowing faster healing.

Tensionable construct 20, 30, 330 can be formed of flexible couplers that are flexible materials and strands such as flat suture, ribbons or flat tape (for example, suture tape) or combination of suture and tape. The flexible strands/couplers may have cross-sections of various forms and geometries, including round, oval, rectangular, or flat, among others, or combination of such forms and geometries. In an exemplary embodiment only, flexible coupler 22 can be suture such as flat suture tape that is braided, knitted or woven. Flexible coupler 22 can be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N. V., Heerlen, the Netherlands), braided with at least one other fiber, natural or synthetic, to form lengths of suture material. Flexible coupler 22 can be braided or multi-filament suture such as FiberTape® suture tape (as disclosed in U.S. Pat. No. 7,892,256, the disclosure of which is incorporated in its entirety herewith) or collagen tape, or wide “tape like” material, or combinations thereof. If suture tape is employed, the tape can have sections with different tapers (for example, 2 or 3 sections of gradual tapers or gradual widths) to facilitate easy formation of the splice regions 55a, 55b and loops 50, 50a, 50b. For example, splice region 55a, 55b (FIG. 11) can be round suture while loop 50a can be formed of flat sections.

Various elements of surgical assemblies 100, 200, 300 can be coated and/or provided with tinted tracing strands, or otherwise contrast visually with the suture ring of the construct, which remains a plain, solid color, or displays a different tracing pattern, for example. Various structural elements of surgical assemblies 100, 200, 300 can be visually coded, making identification and handling of the suture legs and/or loop constructs simpler. Easy identification of suture in situ is advantageous in surgical procedures, particularly during arthroscopic surgeries, such as endoscopy and laparoscopy.

The term “high strength suture” refers to any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

### Example 1

Various construct configurations were tested for tensile fatigue. Construct behavior was analyzed by software to determine effects of the various factors below.

### Methods

A multifactorial design of experiments (DOE) was created in Minitab to evaluate different factors and levels for all suture constructs. Cyclical displacement and ultimate load outcomes were used for factor analysis. The factors and levels included:
1) Sheath (yarn) size: 375 D, 435 D, and 495 D;
2) Length of sheath ring: 26 mm, 33 mm, and 40 mm;
3) Bridge length: 0 mm, 1.5 mm, and 3 mm;
4) Test medium: 20 pcf Sawbone, 40 pcf Sawbone, 80 pcf aluminum plate estimate;
5) Hole size: 3.0 mm, 3.625 mm, 4.25 mm;
6) Deployment tensioning (Hand Tension): 20 lbf, 60 lbf, and 100 lbf;
7) Angle of hole: 0°, 30°, and 60°; and
8) Position of TightRope® construct relative to the end of the loop: 0, 5, 10 (No units, just denoted position as shown in FIG. 21).

20 pcf and 40 pcf Sawbone® blocks were prepared with a mill so that the underside of the block was cut to 0° (i.e., no cutting), 30°, or 60° incline angles. Tunnels were drilled vertically in the blocks from the flat upper surface through the newly cut inclined face. For 30° and 60° inclines, the tunnels created an oblong exit hole. The drill bits used were ø=0.116″, 0.144″, and 0.166″, which respectively approximates the desired 3.0 mm, 3.625 mm, and 4.25 mm diameters. Prior to TightRope® construct insertion, the pre-tensioned diameter of the suture was determined by passing the suture through cannulated anchor spears with known internal diameters. When the diameters were the same and the suture could not be advanced further, the internal diameter was recorded. The TightRope® construct prototypes were inserted into the respective Sawbone® blocks so that the exposed loops were on the upper surface and the anchors were flush with the inclined surface. For samples prepared with the aluminum box fixture, the TightRope® construct prototypes were inserted into the top box face in a 0° orientation. The top box faces had machined 3.0 mm, 3.625 mm, or 4.25 mm hole openings. The box fixtures were placed in an adjustable angle vise to achieve similar 0°, 30°, and 60° inclines. The same gauge length was used regardless of the test media. The exposed suture loop was passed around a dowel rod while the two free ends adjacent to the anchor (used for tensioning) were inserted into a tensioner0 and tensioned to either 20, 60, or 100 lbf. After tensioning, the length, width, and height of the suture anchor were measured via caliper and recorded.

### Tensile Fatigue Testing:

For TightRope® constructs loaded in the aluminum box fixture, the box fixture was angled to either 0°, 30°, or 60° in the adjustable angle vise on the Instron base plate. For TightRope® constructs loaded in a Sawbone® block, an aluminum box fixture secured to the Instron® base plate immobilized the block. Regardless of the testing media, a hook fixture attached to the Instron® load cell was used to apply tension to the constructs. Using WaveMatrix® software, each construct was loaded under tension to 30N followed by a pre-cycle from 10 N—at 1 Hz for 10 cycles. Thereafter, the constructs were loaded to 150 N followed by cycling from 50 N-250 N at 2 Hz for 500 cycles. After cycling, the constructs were pulled to failure at 20 mm/min. The cyclical displacement (not including the pre-cycle) and ultimate load were recorded using Microsoft Excel and Origin software.

### Results:

The data were compiled into a matrixed form, and a DOE analysis was run in Minitab® software. Sample 42 was an extreme outlier regarding cyclical displacement that likely resulted from anchor damage prior to testing. As a result, the observed trends in the DOE were skewed. To combat this, two different DOE screening models were used. The first DOE model eliminated the lowest-performing sample of 3 from each of the 17 groups. The second DOE model replaced the outlier with an average value from the other 2 samples within its group.

The response optimizer was used to set target values, weights, and importance values for desired outcomes—3.5 mm for hand tensioned anchor thickness/height, 600 N for ultimate load, and 2.5 mm for cyclical displacement. The weight and importance for each of these outputs was set at 5, 1, and 1, respectively. Constraints were then added to the response optimizer based on inputs from the intended use of the product—these were 40 pcf test medium (i.e., healthy bone), hole size of 4.25 mm, deployment tensioning of 20 lbf, and 40°-hole angle. The variable ranges for each factor incorporating the constraints and starting values are shown in FIG. 7 and apply to both DOE screening methods.

After response optimization inputs were set, the DOE analysis was run for both screening models, and the ideal solutions were output as a combination of study factors and levels. Additional information (trend charts, factor analyses, etc.) is viewable in the Minitab® files attached to the APT.

TABLE 1
Parameters used in both model's DOE screening response optimizer.
Parameters

| Response | Goal | Lower | Target | Upper | Weight | Importance |
|---|---|---|---|---|---|---|
| Hand Tensioned Thickness/Height | Target | 2.30 | 3.5 | 9.88 | 5 | 5 |
| Ultimate Load [N] | Target | 315.72 | 600.0 | 1423.79 | 1 | 1 |
| Cyclic Displacement [mm] | Target | 0.66 | 2.5 | 4.35 | 1 | 1 |

TABLE 2
DOE solution #1 based on experimental data and response optimizer inputs.
Solution

| | | | | Test | | | | | | | Cyclic | |
|---|---|---|---|---|---|---|---|---|---|---|---|---|
| | | | | Medium | | | | | Hand | | Displace- | |
| | Sheath | Length of | Bridge | (Foam | Hole | Deployment | Angle | | Tensioned | Ultimate | ment | |
| | (Yarn | Sheath | Length | Block, | size | Tensioning | of hole | Position of | Thickness/ | Load [N] | [mm] | Composite |
| Solution | Size) | Ring(mm) | (mm) | Flat P | (mm) | (LBF) | (deg) | TR Routing | Height Fit | Fit | Fit | Desirability |
| 1 | 385.394 | 34.6263 | 3 | 40 | 4.25 | 20 | 40 | 9.94510 | 3.50199 | 607.327 | 2.67999 | 0.983181 |

TABLE 3
DOE solution #2 based on experimental data and response optimizer outputs.
Solution

| | | | | Test | | | | | | | Cyclic | |
|---|---|---|---|---|---|---|---|---|---|---|---|---|
| | | | | Medium | | | | | Hand | | Displace- | |
| | Sheath | Length of | Bridge | (Foam | Hole | Deployment | Angle | Position of | Tensioned | Ultimate | ment | |
| | (Yarn | Sheath | Length | Block, | size | Tensioning | of hole | TR Routing | Thickness/ | Load [N] | [mm] | Composite |
| Solution | Size) | Ring(mm) | (mm) | Flat P | (mm) | (LBF) | (deg) | Relative | Height Fit | Fit | Fit | Desirability |
| 1 | 375 | 26 | 0.0227586 | 40 | 4.25 | 20 | 40 | 3.63636 | 3.49983 | 560.508 | 2.63897 | 0.967498 |

### CONCLUSION

In this study, the DOE solutions were constrained to specific factor levels to approximate outcomes based on proper clinical intended use. A 40 pcf Sawbone® model was chosen since it simulates good bone quality. A hole size of 4.25 mm was selected because that is the drill bit size that can be used for tunnel creation. A deployment tensioning of 20 lbf was selected because it approximates hand-tensioning load and the use of a tensiometer (i.e., for higher tensioning loads) is not desired in clinical applications.

Target values were selected in the DOE response optimizer based on a preliminary analysis of the data and by input from product management on what is deemed clinically acceptable. The DOE solution was weighted in a 5:1:1 ratio for thickness/height to ultimate load and cyclical displacement. The higher weighting for thickness/height signifies high priority in minimizing implant prominence. The composite desirability for the DOE was high (>0.96) for both models.

Two different models were analyzed to address the presence of outliers. Though the methodology to eliminate outliers was different, the two models were largely convergent.

What is claimed is:

1. A surgical construct comprising suture comprising a hollow core and 52 picks per inch or less in an enclosed ring, wherein the construct is configured to fold into a “S”, “U”, or “W” configuration.

2. The surgical construct of claim 1, wherein the suture has 40 picks per inch or less.

3. The surgical construct of claim 1, wherein the suture has 25 picks per inch or less.

4. The surgical construct of claim 1, wherein the suture has 15 to 20 picks per inch.

5. The surgical construct of claim 1, wherein the suture has 18 picks per inch.

6. The surgical construct of claim 1, wherein the suture has 32 yarns or less.

7. A method of forming a ring of suture comprising:
a) inserting passing tail 1 into splice point 4 on side A;
b) direct passing tail 1 to splice point 3;
c) exiting passing tail 1 at splice point 3 on side B;
d) inserting passing tail 2 to splice point 3 on side B;
c) direct passing tail 2 to splice point 3;
f) exiting passing tail 2 at splice point 3 on side B,
g) wherein steps (a)-(f) form a ring; and
h) folding ring into 3 sections to form a “S”, “U”, or “W” configuration.

8. A surgical assembly for tissue repairs, comprising:
a fixation device in the form of a flexible ring including a suture with a hollow core, wherein the suture has a pick per inch of 52 or less, and a yarn count of 32 or less; and
a tensionable construct loaded onto the fixation device.

9. The surgical assembly of claim 8, wherein the fixation device consists essentially of ultrahigh molecular weight polyethylene suture and at least one radiopacifier.

10. The surgical assembly of claim 8, wherein the fixation device consists essentially of ultrahigh molecular weight polyethylene suture and bismuth trioxide.

11. The surgical assembly of claim 8, wherein the tensionable construct comprises a flexible coupler and at least one closed, adjustable, continuous, flexible loop having an adjustable perimeter.

12. The surgical assembly of claim 11, wherein the flexible coupler is suture or suture tape.

13. The surgical assembly of claim 11, wherein the tensionable construct comprises two closed, adjustable, continuous, flexible loops, and a loop interconnection.

14. The surgical assembly of claim 8, wherein the tissue repair is rotator cuff repair, AC joint repair, syndesmosis repair, Achilles tendon repair, patellar tendon repair, ACL/PCL reconstruction, hip and shoulder reconstruction, AC joint reconstruction, syndesmosis reconstruction, quad/patellar tendon rupture repair, or hallux-valgus repair.

15. The surgical assembly of claim 8, wherein the flexible ring is configured to fold into a “S”, “U”, or “W” configuration.

16. The surgical assembly of claim 8, wherein the flexible ring is formed by:
providing a suture strand with a first end terminating in a first passing tail, and a second end terminating in a second passing tail;
inserting the first passing tail into a first splice point, to a second splice point and exiting the suture strand at a third splice point;
inserting the second passing tail into the third splice point, to the second splice point and exiting the suture strand at the first splice point; and
trimming the first passing tail and the second passing tail up to a next splice point.
